(19) United States
(12) Patent Application Publication
(10) Pub. No.: US 2004/0019292 A1
(43) Pub. Date: Jan. 29, 2004

(54) Method and apparatus for bioelectric impedance based identification of subjects

(76) Inventors: Darrel Dean Drinan, San Diego, CA (US); Carl Frederick Edman, San Diego, CA (US)

Correspondence Address: Darrel Drinan, Suite 152, 11772 Sorrento Valley Road, San Diego, CA, 92121, US

(21) Appl. No.: 10/628,987

(22) Filed: Jul. 28, 2003

Related U.S. Application Data
(60) Provisional application No. 60/399,353, filed on Jul. 29, 2002.

(51) Int. Cl. A61B 5/05

(52) U.S. Cl. 600/547

(57) ABSTRACT

The use of this invention relates to the bioelectric impedance method and associated apparatus and systems for the purpose of identification. The bioelectric impedance identification system is comprised of apparatus for generating bioelectric impedance data, storage for storing bioelectric impedance data and comparators for determining whether one set of bioelectric impedance data is the same or different than another set of bioelectric impedance data wherein said sets of data may arise from one or more subjects. The invention has utility in applications in the area of governing access, and the control of this access, e.g. to logic circuitry or control units, (e.g. computers, local area networks, or video game joystick control apparatus), “smart” credit cards (cards containing integrated circuits or chips) or access to facilities, equipment or storage apparatus, (e.g. machinery, rooms, garages or cabinets).

### CROSS-REFERENCE TO RELATED APPLICATIONS

This application claims the benefit of U.S. Provisional Application Ser. No. 60/399,353, filed Jul. 29, 2002, which is incorporated herein by reference.

### FIELD OF THE INVENTION

This invention relates to the methods and apparatus for the identification of subjects. More particularly, it relates to the use of bioelectric impedance as a means of identification. The invention provides for methods and apparatus to measure bioelectric impedance values from one or more subjects and the method and apparatus to compare one or more measurements values, either by direct comparison of these data or by the modification of these data to allow for variation. In addition, the invention provides for the method and apparatus to provide local or a remote comparison to permit a subsequent response based upon the comparison.

### BACKGROUND OF THE INVENTION

Rapid, secure unique identification is a rapidly growing global need. Needs range from financial transactions, access to facilities and information systems and to everyday activities, e.g. use of electronic games, entrance to cars or housing. A number of biometricbased solutions exist, either as stand alone tools or in combination with other parameters, to provide enhanced unique identification. These include: fingerprints; retinal scans; voice recognition and facial morphology. One problem these systems have is that, by themselves, each is unable to provide a high level of certainty in the identification process. That is, they may not ensure the “lively-ness” of the measured object, e.g. the use of substitute materials to compromise fingerprint scanners, or they may be compromised by other external factors.

In order to provide greater certainty in identification, multiple systems are frequently combined, e.g. bankcards with personal identification (PIN) numbers, identification badges with PIN numbers, fingerprint and identification badges. However, this combination may require expensive additions and modifications to the identification means. What is needed is a inexpensive, fast, and accurate identification means that will provide either a certain level of identification certainty by itself or when combined with additional methodologies, will provide an increase in identification certainty.

Electrical measurements have been employed as a means of obtaining fingerprint patterns. For instance, Knapp (U.S. Pat. No. 5,325,442) discloses a fingerprint sensor that, in essence, is multiple capacitors formed when the overlying fmger comes into contact with underlying plurality of electrodes. Such methods are well known to those skilled in the art but each will have limitations and drawbacks, e.g. Salatino et al., (U.S. Pat. No. 5,862,248) noting that the electrode spacing in Knapp may have a finite resolution due to possible effects between adjacent electrodes.

However, one area employing electrical measurement that has not been extensively utilized for the purposes of identification is the impedance of a body (or portion thereof) to an introduced electrical signal. In general, impedance is the degree to which electronic components impede the flow of current and is a frequency-dependant quantity. Bioelectric impedance refers to (human or animal) being represented as an electronic circuit having impedance, and may include both resistance and reactance measurement at one or more frequencies.

For instance, Setlak (U.S. Pat. No. 5,953,441 & U.S. Pat. No. 6,067,368) teaches that bioelectric impedance may be used in conjunction with a fingerprint scanner in order to validate that a live fmger, but not an image or other material, is being utilized in operation of the scanning apparatus. The purpose to which the wide range of phase angles are employed in said inventions is for solely for the purposes of discriminating between a live fmger versus fake, and not for the purpose of individual identification.

In a non-identification need related application, bioelectric impedance is widely used in the healthcare industry for the calculation of body fat percentage or body hydration (total body water). Such apparatus typically need to employ an algorithm including factors such as age, sex, height and weight, in addition to the measured bioelectric impedance in order to determine the body's hydration or fat percentage. Such modifying factors are required to remove and adjust for individual variations in measured bioelectric impedance in order to determine the desired parameter, e.g. hydration or fat percentage, irrespective of the individual's personal bioelectric impedance readings. In addition, it has been noted that intra-individual bioelectric impedance values are less variable than inter-individual values. Thus, it may be further inferred that bioelectric impedance readings are, in general, are highly individualistic and therefore may be employed for the purposes of identification.

For instance, Brooks (U.S. Pat. No. 6,507,662 & U.S. Pat. No. 6,343,140) teaches the use of transforming the bioelectric impedance data, e.g. conversion of the impedance data obtained from multiple body segments to a ratio or conversion of impedance data to a multidimensional matrix representation. This transformation is done prior to use for the purpose of identification in order to account for possible variations in measurement position or variations in bioimpedance, e.g. diurnal variation. However, such conversions may result in loss of sensitivity of the identification process, e.g. use of a ratio may result in overall reduction of information whereby two or more pieces of data are compressed into a single value or that different individuals may have similar ratios, e.g. a small child and large adult. In addition, such mathematical means are not requisite for the purpose of bioelectric impedance identification when other means may be employed to resolve bioimpedance variation, e.g. trend analysis, guides for location of measurement on body, etc.. Therefore, there remains a need to provide additional efficient and accurate means of identification as well as alternative means to utilize bioelectric impedance for the purpose of identification.

### SUMMARY OF THE INVENTION

The use of this invention relates to the bioelectric impedance method and associated apparatus and systems for the purpose of identification. The bioelectric impedance identification system is comprised of one or more apparatus for generating bioelectric impedance measurement data, storage for storing bioelectric impedance data and comparators for determining whether one or more sets of bioelectric impedance data is the same or different than another set of bioelectric impedance data wherein said sets of data may arise from one or more subjects. The apparatus used for the system comprise the hardware and software required to enable the bioelectric impedance identification system.

Bioelectric impedance may be employed as a system of identification either by itself or in combination with other identification tools, e.g. RF (radio frequency) proximity identification cards, fingerprint scanning, hand morphology scanning, iris scanning, code words/numbers, etc.. In particular, key aspects of the bioelectric impedance measurement system, e.g. electrodes, may be integrated into other apparatus or systems, e.g. a keyboard (or other form of input/output apparatus), in order to facilitate identification of a subject utilizing the apparatus. The purpose of identification in such interfaces may be to regulate, or to define levels of access, or use of the apparatus. Access in general includes, but is not limited to, access to logic circuitry or control units, (e.g. computers, local area networks, or video game control apparatus such as joysticks), “smart” credit cards (cards containing integrated circuits or chips) or access to facilities, equipment or storage apparatus, e.g. rooms, garages or cabinets. The term access in this context may also be used to either allow or prevent subsequent activity on the part of the subject. In addition, bioelectric impedance may be used as part of keyless entry systems, e.g. radiofrequency (RF) inductive identity cards or garage door openers having bioelectric impedance apparatus.

In other aspects of the invention bioelectric impedance method may employ, for purposes of comparison, minimal modification of bioelectric impedance data or with modifications of data values. Such modifications may include, but are not limited to, the use of multiple reference measurements taking over time to establish normal range of readings or by the use of trend analysis based upon multiple bioelectric impedance values taken over extended periods of time to adjust reference points in correspondence with the data or time of measurement. In addition, additional values or factors, e.g. weight, may be employed to adjust bioelectric impedance values directly or for reference measurement adjustment.

In operation, the impedance of an individual is measured at one or more frequencies, creating a data set for each individual. This data set may be compared to one or more stored reference templates to establish an individual's identity. Having an independent measurement of parameters derived from the subjects body's characteristics reduces significantly the possibility of fraudulent use, or compromise of the identification process and improves identification accuracy. The use of bioelectric impedance measurement (at one or more frequencies) for the purpose of identification verification therefore represents a novel means of identification, useful either by itself or in combination with other identification technologies.

Three system activities are necessary elements of this invention. The first activity is the enrollment or recording of one or more subjects bioelectric impedance values into a reference template (reference). Such an activity creates a reference template database for subsequent identification comparisons. The second activity is the measurement of one or more subject's bioelectric impedance values by a bioelectric impedance measurement apparatus during an identification need activity (query). These two activities may or may not be sequential, i.e. a query may be executed prior to the recording of that individual's bioimpedance data into a reference template database. The third activity is the comparison of bioelectric impedance data obtained from the query to the reference template data present in the data base and an assessment as to whether the query data matches or is concordant with reference template data (assessment). Each of these activities may utilize a number of different forms based upon the identification application. The scope of this invention is not restricted to any one application or form.

One requisite activity for implementation of this invention is the entering a subject's bioelectric impedance data into a reference template database. Such bioelectric impedance data may be obtained from one or more frequencies of applied electrical signal and involve one or more locations upon the subject's body. This may be done at one or more pulsed DC or AC frequencies. These frequencies are preferably chosen between 5 KHz to 250 KHz, and more preferably from the frequencies from 5 KHz to 10 OKHz and 50 KHz to 60 KHz. The measured bioelectric impedance to the introduced signal, includes, but is not limited to, body resistance (or voltage drop at known current) and body capacitance (phase shift of introduced signal). Those skilled in the art of bioelectric impedance will recognize that the equipment and methods to introduce one or more electrical signals into the body, or portion thereof, are well known.

As deemed necessary for the needs of the identification process, other information, e.g. time/date of measurement acquisition, driver's license, photo, passport information, or other biometric data, such as fingerprint pattern, iris pattern, facial geometry, weight, height, etc., may be additionally entered into the reference template data base.

In one form of the invention, a subject engages a bioelectric impedance measurement identification system constructed primarily for the purpose of identification, e.g. a door access apparatus. In use, the subject contacts the signal introduction and measurement electrodes and has their bioelectric impedance measured (query). This measurement is then compared (assessment) to a reference template (reference). Based upon the comparison algorithm that may include boundaries, trend analysis or modification factors that may be stored with the data present within the reference template data, a variety of assessment outcomes are possible.

Assessment outcomes include, but are not limited to, deciding whether the subject is present or not present within the reference template data set. The outcome may permit subsequent responses to the identification system, including, but not limited to, initiating access or denial of access, e.g. to a facility, computer system or fmancial transaction. It is understood that the query and assessment activities may be conducted at the same or multiple, remote locations, including but not limited to communication between the reference template data base, the assessment and the query location occurring by wireless means.

It is understood that the site of electrode placement on the body as well as the electrical signals employed for the purpose of query will either be the same as those for the reference template data base, a subset of the reference template, or be readily correlated to the reference data base template values. In addition, a variety of other factors may be included in the reference template data base, including, but not limited to, a subject's weight and/or height. These factors may be obtained while they are utilizing the bioimpedance measurement apparatus, i.e. they may stand on a scale or have their height measured while grasping/holding/touching the electrodes. These values, in combination with the bioelectric impedance reference template data, may provide a higher degree of identification or uniqueness to the individual than the bioelectric impedance template alone.

In addition, subject's bioelectric impedance template may be adjusted or tolerances included to account for changes in response due to the body's hydration status, supine versus upright posture, ambient environment, elevation, time of day, etc.. Because body composition may change over periods of time, another aspect of this invention may include the means to track changes in bioelectric impedance values for an individual and to adjust or provide adjustment factors to the corresponding reference data base template. These adjustment factors may be based on data forthcoming from reference template data creation or queries, on one or more occasions, e.g. daily or weekly.

### Alternate Embodiments

1. The use of bioelectric impedance on apparatus whose secondary purpose is that of identification. In such apparatus, electrodes are positioned in such a fashion to be substantially located on the surface of said apparatus, e.g. on keys of a keyboard, as contact points on a surface of a keyboard, on one or more sides of a “smart card” or on a joystick/game controller. In applications such as use with computer keyboards, two hands may be employed for the purpose of enabling the electrical signal to pass through the body, e.g. electrodes positioned on computer keyboard keys (or on adjacent surface space of the keyboard). In still other variations of this application, only one body segment, e.g. a hand, is in contact with the bioelectric impedance measuring electrodes. Other variations may employ within segment (e.g. within hand) measurements. In these alternate applications of the invention, the subject places a single segment, e.g. a hand, in contact with the bioelectric impedance current and measurement electrodes. These electrodes, positioned on the surface, then are employed to obtain the bioelectric impedance measurements. Other electrodes placements include, but are not limited, finger to finger measurements on one hand, finger to wrist measurement or within palm multiple site measurements. Applications may include the use of computer game joysticks, computer mouse, “smart cards” or other such apparatus whereby for convenience a single hand or other single segment of the body is more commonly employed in manipulating the apparatus.

2. The use of a plurality of reference measurements taken for the purpose of providing the basis for adjustment of the reference template data and/or assessment parameters. Such adjustments may be based upon factors including, but not limited to: temporal trends in bioelectric impedance measurements, e.g. daily, weekly or monthly variations; altered physiological status, e.g. weight gain; or measurement variation, e.g. variations in placement of the hand upon the measurement apparatus.

3. The use of bioelectric impedance measurement apparatus whose electrodes remain in substantial contact with the subject. Such apparatus may include those apparatus whose primary or secondary function is that of identification. Such apparatus may include apparatus that have as their primary purpose that of monitoring physiological function, e.g. heart rate, blood oxygen and temperature monitoring straps, or patches. The bioelectric impedance electrodes are effectively continually in contact with the surface of the body, e.g. present for periods of time not during bioelectric impedance measurement and may take the form of adhesive patches or strap containing electrodes positioned upon the body or body segment.

4. The use of electrodes in contact with body surfaces other than hands.

5. The use of conductive patches or other electrodes affixed to the body by other methods, e.g. a strap, and having apparatus suitable for receiving an instructional signal without the necessity of direct physical contact between the subject and a bioelectric impedance signal measurement control source. This would include, but not be limited to, radio waves, infrared signals or acoustic signals. These signals would instruct one or more electrodes to send and measure one or more bioelectric impedance electrical signals to one or more other electrodes placed on the body. The resultant bioimpedance template data is then be sent from the receiving electrode(s) in a wireless fashion to a suitable reception station. This data may then be compared to template values stored within a reference template database. This would enable the subject to pass through a signal generating station and have their bioimpedance values determined in a remote and possibly mobile fashion. To enable this aspect, the circuit loop between electrodes may be closed by the use of a wire or other conductive material or the body itself (e.g. by clasping hands) may provide the appropriate loop.

6. The signal used to generate the bioelectric impedance template may be obtained from a pulse of electricity or burst of frequencies and the measured values obtained by signal de-convolution rather than utilizing a sequence of one or more defined frequencies being read in a serial fashion.

7. The electrical signals used for the generation the bioelectric impedance values of a subject may be a subset of those frequencies used to construct the reference template database. The subset of frequencies chosen may be fixed or randomized. By use of a subset of frequencies, time and instrumentation costs may be significantly reduced during the point of identification query.

This invention may be embodied in many different forms and should not be construed as being limited to the embodiments described above. Those skilled in the art will readily understand the basis and means of the invention as described by the embodiments.

### BRIEF DESCRIPTION OF THE DRAWINGS

FIG. 1—Schematic portrayal of the relationship between the three elements of bioelectric impedance identification system.

FIG. 2—Block diagram of one embodiment of components of an apparatus to obtain bioelectric impedance data.

FIG. 3—Illustration of one embodiment of an apparatus surface for obtaining within hand bioelectric impedance measurements.

FIG. 4—Ranked bioelectric impedance data from 250 individuals.

FIG. 5—Bioelectric impedance and phase angle data from 250 individuals.

FIG. 6—Example keyboard showing one possible set of electrode placements for bioelectric impedance measurements.

### DEFINITIONS

1. Bioelectric Impedance—Bioelectric impedance is a body (human or animal) representated as an electronic circuit having both resistance and reactance. Measurements obtained from bioelectric impedance may include impedance or phase angle at one or more signal frequencies.

2. Bioelectric Impedance Analysis—The use of one or more frequencies, preferably between 5 KHz to 250 KHz, to measure either phase or amplitude changes in the input signal resultant from the subject's body electrical response characteristics.

3. Impedance—Impedance is the degree to which an electronic component impedes the flow of current. In general it is a frequency-dependant quantity. The impedance of a resistor is also called its resistance. The impedance of capacitors and inductors is also called their reactance.

4. Impedance ValuesThe resultant impedance data including resistance and/or capacitance or the calculated impedance value(s) and/or phase angles obtained from one or more bioelectric impedance measurements at one or more frequencies or currents that is subsequently utilized for the purpose of identification.

5. Reference Template—Bioelectric impedance values and possibly other factors, e.g. height, weight, fingerprint data, sex, utilized for identification of a subject.

6. Segment That portion of a subject's body through which a bioelectric impedance signal is passed. A segment may comprise effectively the entire subject, e.g. hand to foot measurements, or a portion thereof, e.g. within hand measurements, hand-to-hand measurements, thigh measurements, thoracic measurements or other body region measurements.

7. Subject A human or mammalian participant who undergoes bioelectric impedance measurement.

### DETAILED DESCRIPTION OF THE INVENTION

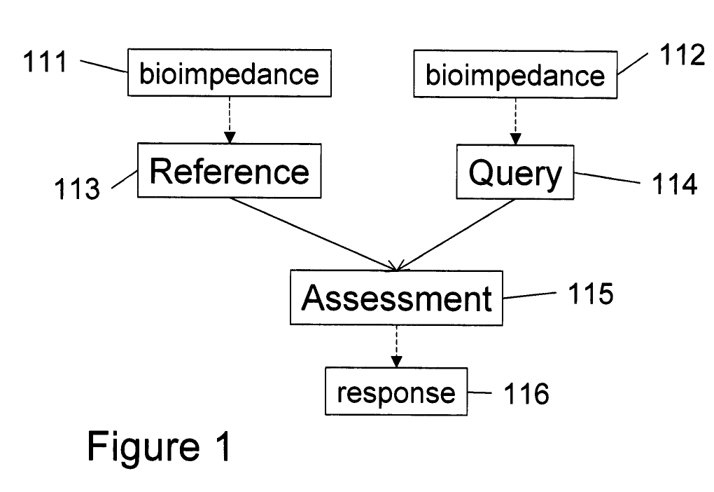

The present invention will now be described more fully with reference to the accompanying drawings in which aspects of the invention are shown. The system of bioelectric impedance identification requires three elements. These three elements are: a) the reference template data creation; b) the query; and c) the assessment. In FIG. 1, the relationship of bioelectric impedance measurements (111,112) the creation of the reference template data (113), the query for purpose of identification (114) followed by assessment of the query's inclusion or exclusion from the reference template (115) and the response to the identification system (116) is diagrammatically portrayed. Each aspect of this invention is described in more detail below with the understanding that one or more forms of each may be combined with one or more forms of each of the others to enable the invention.

### System Elements—Reference Data (Measurement, and Storage)

The identification of presumptively unknown subject requires the measurement, and storage of one or more bioelectric impedance values, or portion thereof, of one or more subjects. For the purposes of description, the measurement of impedance and the calculation of impedance based on introduced and measured electrical signals are used interchangeably throughout. Such data stored as templates for this purpose of subsequent comparison are defined as reference (113) or reference template data. One or more such reference templates may be used for the purpose of assessment at times of identification need. The process of constructing a reference template is termed a reference process and the apparatus useful for obtaining such reference bioelectric impedance data is a reference bioelectric impedance apparatus. Implementation of a reference process requires the measurement using one or more such reference apparatus and the subsequent storage of resultant values, including but not limited to bioelectric impedance data, into a data storage system or storage apparatus.

In one aspect of the invention, one or more reference measurements may be taken for the purpose of constructing a set of bioelectric impedance data associated with temporal or other factors, including but not limited to, the date or time of measurement, physical parameters, such as weight, measurement variation, etc. That is, a first set of reference bioelectric impedance data is taken and stored. A second (or additional) set of reference bioelectric impedance data may be added a later time(s). In this context, a set of bioelectric impedance data refers to those measurements taken from one or more locations on the body at one or more electrical signals during a reference process event. A reference template for a subject may be comprised of one or more sets of reference data. Multiples sets of bioelectric impedance data sets within a template may provide a basis for subsequent pattern or trend analysis during the assessment process, thereby allowing adjustment of template values in accordance with such pattern or trend analysis.

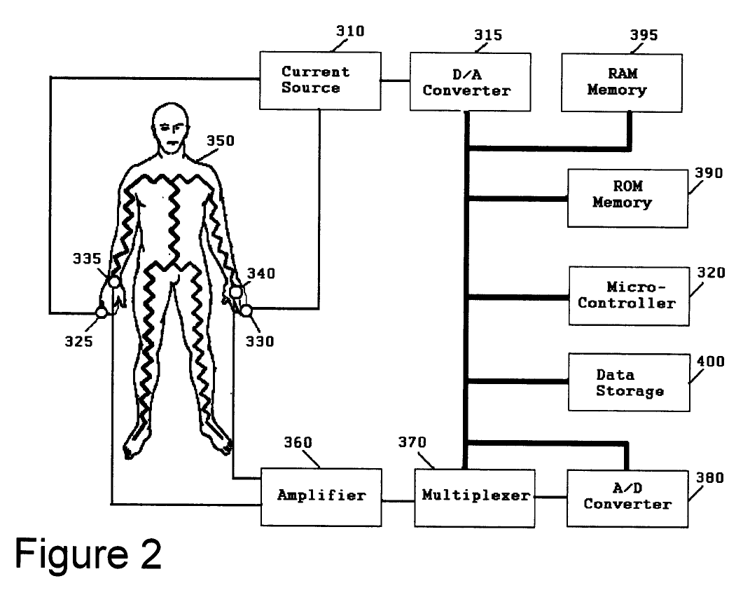

The obtaining of one or more bioelectric impedance values (or data) requires the introduction of one or more electrical signals into a subject's body. This requires both the means to generate one or more electrical signals and a means to introduce this signal into the body. The form of electrical signal is preferably AC in nature, and chosen from the frequencies between 100 Hz and 1 MHz and preferably, one or more frequencies between the frequencies of 5 KHz to 250 KHz is selected. FIG. 2 illustrates one embodiment of components for a bioelectric impedance system for obtaining bioelectric impedance data. Other designs and structures that perform this task are also possible and this embodiment is not intended to limit the scope of this invention.

In operation, one form of delivery of said electrical signal to the body is by sourcing current (310) to a predetermined value preferably between 10 microamperes and 10 milliamperes and more preferably between 100 microamperes and 1 milliamperes. However, this invention is not restricted to sourcing current but may also include other variations such as sourcing voltage or power. The generators of such an electrical signal, e.g. a frequency generator, are well known to those skilled in the art of bioelectric impedance measurement. The preferred method of generating this electrical signal is within the logic core or microcontroller (320). The digital output of the microcontroller may pass through a digital to analog converter (315) in order to control the current source (310).

Electrically conductive structures, e.g. a plurality of electrodes (325, 330, 335, 340), may be used to introduce the electrical signal, e.g. current, into a subject such that a circuit is made between a least two signal introduction electrodes passing through the subject or portion of the subject's body (325, 330). Such conductive structures may include, but are not limited to, electrically conductive metals, sintered metallic composites, polymers, gels, carbon-based systems, silicon apparatus, electrically conductive microneedles, or conductive solutions. In a one form of the invention, the electrodes consist of a conductive metal constructed from stainless steel or a gold coated material.

These electrodes are constructed in such a fashion that the user's body surface comes into contact with the electrodes at one or more locations on the subject's body. The body surface includes the body surfaces covered by epidermis or other related cell types and exposed to the external environment, either continually or transiently. Examples of these surfaces include but are not limited to: skin or internal surfaces such as the mucosal surfaces found in the mouth, nasal passages or other body passages or orifices. In use, the electrodes are positioned such that the circuit formed passes substantially through one or more segments of the users body (350).

In order to ensure that the subsequent bioelectric impedance measurement involves the interrogation of tissue substantially beneath the body surface, such electrically conductive structures, e.g. electrodes, used to introduce the electrical signal shall be placed further apart than approximately ten times the thickness of the skin, i.e. greater than 2.5 millimeters, or shall penetrate the body surface, e.g. microneedles through the skin. The basis for this restriction is that a body surface such as the skin has substantially higher impedance, approximately tenfold, than underlying tissue and therefore the conductive pathway from sources farther apart than ten times the thickness of the body surface (skin) follows, in large part, the lower impedance of the tissue underlying the surface between these points. Tissue includes, but is not limited, body fluids including both intracellular and extracellular fluids, various cell types such as adipocytes, fibroblasts, and muscle cells, as well as connective structures. In one embodiment of the invention, the distance between any two conductive elements used for introduction of the electrical signal is least 1 cm.

In one form of the invention, the user places each hand upon two different surfaces, with each surface containing one or more conductive structures (electrodes) for the purpose of electrical signal introduction and measurement. In one embodiment, the conductive means for signal introduction are different than those for measurement, e.g. a four electrode system may be employed with two electrodes for signal introduction and two electrodes for measurement, with one signal and one measurement electrode for contact with each hand. The circuit thus formed passes substantially through each arm and the upper trunk region.

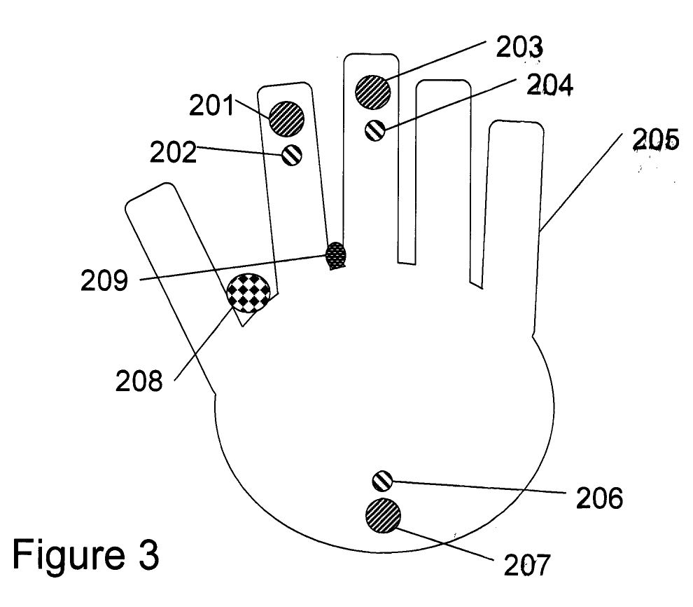

In an alternate embodiment of the invention, the electrodes may be positioned such that the current passes substantially within one hand. FIG. 3 illustrates one such alternative embodiment. The outlined hand (205) describes the apparatus surface location where a subject places one hand. Shown also are the electrically conductive structures (e.g. electrodes) for signal introduction, (201,203, 207) and signal measurement (202, 204, 206). Having a plurality of signal introduction and measurement structures permits multiple signal measurement possibilities, e.g. index finger to middle finger, index finger to palm and middle finger to palm and thereby increases the flexibility and quality of the identification process.

In an alternate form of the invention, the electrically conductive structures (electrodes) for electrical signal introduction and measurement may be located on opposing surfaces of the measurement apparatus. One such embodiment is the signal introduction electrodes may be located on top surface and measurement electrodes may be located on the bottom surface of a “smart card”. In alternate forms of the invention, they may be located on the same surface, e.g. a plurality of electrodes being located on the one side of the “smart card”. In use, the subject contacts the electrodes either using a single hand or with both hands, e.g. holding the card between the thumb and forefinger of both hands, dependent on design. Other designs and structures that perform this task are possible and these embodiments are not intended to limit the scope of the invention.

Returning to the embodiment shown in FIG. 2, the current passing through the segment causes a voltage drop proportional to the tissue impedance. This resultant voltage is measured using electrically conductive structures in contact with the body, e.g. measurement electrodes (335, 340), and passed to sense amplifier (360) where it is amplified and generally filtered to remove signal noise before being passed to the analog multiplexer (370). The multiplexer passes the bioelectric impedance measurement signal as well as other analog signals, e.g., temperature, if included, to the A/ID converter (380). The timing of the voltages sampled and being sent to the A/D converter is controlled by microcontroller (320). For the purpose of bioelectric impedance measurement, the electrically conductive structures used for the purpose of measurement, e.g. sense electrodes, may be the same or different conductive structures used for introduction of the bioelectric impedance signal. In addition, these measurement structures may have the same or different form or composition as compared to the structures used for introduction of the bioelectric impedance signal.

Logic operations and mathematical calculations, e.g., impedance, may be controlled by a software program stored in the program ROM (390). Temporary storage for these calculations can be provided in the RAM (395). For signal analysis processing from a multifrequency electronic signal, both electronic components, e.g. filters, and mathematical operations, such as fast Fourier transformation, may be employed to obtain bioelectric impedance data. Numerous alternatives exist for the calculation and control of the bioelectric impedance measurements and are well known to those skilled in the art of electrical engineering.

It is desired that the bioelectric impedance signal being measured will reflect the impedance of tissue substantially beneath the body surface between the measurement structures. As with the signal introduction structures, in order to ensure that the measurement involves the interrogation of tissue substantially beneath the body surface, such measurement structures shall be placed further apart than approximately tenfold the thickness of the skin, i.e. greater than 2.5 millimeters, as is required for the signal introduction conductive means. In a preferred embodiment of the invention, the distance between any two measurement conductive means is least 1 cm.

In a further refinement of the invention, two or more electrically conductive measurement means, e.g. electrodes, may be positioned in such fashion as to be substantially within the field generated by the source current, e.g. the two or more measurement electrodes are positioned on the body substantially between two or more signal introduction electrodes.

One additional aspect of the invention is that the bioimpedance measurement apparatus, whether designed for an extremity, e.g. hand to hand, or other segment of the subject, may be constructed in such a way as to provide repeatability of the measurement. Such repeatability may be obtain by means of mechanical guides or fittings, e.g. posts, slots, spring based compression structures or ledges. FIG. 3 illustrates one embodiment of mechanical guides for locating a hand upon a bioimpedance measurement apparatus surface. As shown in FIG. 3, positional guide posts (208, 209) are positioned to aid in the placement of a subject's hand upon both signal introduction (201, 203, 207) and measurement (202, 204, 206) electrically conductive structures (electrodes). Other designs and structures which perform this task are possible and this embodiment is not intended to limit the scope of the invention.

In still other embodiments of the invention, non-mechanical means may be utilized to provide data to correct for subject inconsistency in contacting the measurement apparatus. Such means may include, but are not limited to, sense electrodes determining body segment position or location on the apparatus, visual inspection or finger print systems providing placement data or mathematical algorithms sensing electrode surface contact area and thereby providing a means for bioelectric impedance measurement correction. Such systems providing such data may include, but are not limited to, fingerprint and hand geometry scanners.

An apparatus, or portion thereof, constructed for the purpose of query or reference bioelectric impedance template measurement may include, as part of its operation, the ability to provide feedback to the subject indicating the degree of contact between the body and the electrically conductive elements as well as the completion (or lack thereof) of bioelectric impedance measurements. Such feedback may include, but is not limited to, tactile (sensing a key click, or weak electrical current), audible (a beep or chime), or visual (such as a series of lights, e.g., red, yellow, green, indicating quality of contact or degree of measurement completion) methods.

In yet other embodiments of the invention, multiple sets of electrodes may be utilized to provide multiple impedance readings corresponding to different geometries/contact points of the electrode arrangement. An example of this embodiment is a hand bioelectrical impedance measurement apparatus wherein each finger (and/or palm) is in contact with one or more electrodes such that measurements would be obtained of the span between the first and second finger, first and third finger, second and fourth finger, and so on. The sites, sequence and timing of such measurements may also be included in the overall bioelectric impedance measurement data. Such multiple measurements may improve the overall quality of the identification process by providing additional data for analysis and comparison.

In still other embodiments of the invention, the electrodes are substantially in contact with one or more portions of a body, e.g. on the wrist, thigh, trunk or ankle, for an extended period of time, e.g. a period of time longer than that required to obtain one or more bioelectric impedance template measurements. In this embodiment of the invention, the electrically conductive structures, e.g. electrodes, are positioned substantially on one aspect of a solid surface, e.g. an adhesive strip, patch or on a strap, or otherwise supported, e.g. by a spring or lever, and are thereby being held in contact a subject's body. Such apparatus may consist of principally electrically conductive structures with the requisite power, control and measurement circuitry located in an adjacent apparatus wherein contact between the electrically conductive structures and the adjacent apparatus is by wires or other physical linkage. In an alternative embodiment, a apparatus in substantial contact with the body surface includes not only the electrically conductive structures, but also contains a power source, antenna, signal generation and measurement circuitry, microcontroller, as well as additional communication linkage, if required. Communication with such an apparatus may use wireless, e.g. RF, IR or acoustic, or wired (physical) links. Such apparatus whether located adjacent to the body or substantial contact with the body surface may also include display (e.g. LCD) and input methods (e.g. keyboard) to increase flexibility and usefulness.

As noted earlier, bioelectric impedance reference template may be created by use of one or more frequencies selected from the range of 100 Hz to 1 MHz. In one embodiment of the invention, the subject's bioelectric impedance reference template is resultant from a plurality of frequency bioelectric impedance measurements, with at least one frequency chosen from the frequencies between 1 KHz and 20 KHz and at least one frequency selected from the range of 50 KHz to 250 KHz. Low frequency signals, e.g. 5 KHz, are believed in the field of bioelectric impedance measurement to be representative of extracellular conductivity whereas higher frequency measurements, e.g. >50 KHz, are believed representative of both intra and extracellular conductivity. Thus, by inclusion of signals representative of both ranges, more detailed bioelectric impedance reference template data of a subject may be created. However, in alternate embodiments of the invention, one or more frequencies may be selected from the range of frequencies, between 100 Hz to 1 MHz. These bioimpedance data from these or other embodiments of the invention may then be employed in the creation of a subject's bioelectric impedance reference template.

As noted earlier, bioelectric impedance reference template refers to bioelectric impedance data used subsequently in the assessment process for the purpose of identification. In a one embodiment of the invention, the term “bioelectric impedance reference template” refers to unaltered or unmodified measured bioelectric impedance data that is subsequently used for the purpose of comparison. In alternative embodiments of the invention, bioelectric impedance data may be modified in some mathematical fashion, e.g. to adjust to long term changes in body composition, or combined with additional data such as finger print data, PIN numbers, badge identification, etc., to provide a basis for subsequent assessment.

In one form of the invention, data modification includes the methods to minimize noise or other similar effects arising during the measurement process. Such methods include, but are not limited to, the averaging of several measurements taken rapidly, e.g. within one second, and repetitively at one frequency, to provide an average signal value or the discarding of one or more repetitive measurements if the value is beyond some predetermined out of range value, e.g. greater than two standard deviations from the mean value of multiple measurements.

After initial reference template data is created, e.g., bioelectric impedance measurements at different frequencies, this data plus other biometrics, numeric codes, answer to questions, etc. if included, may be stored (400) for future assessment. Storage may be of a variety of means, including, but not limited to, portable local, stationary local, or remote. Portable local data storage refers to one or more portable apparatus that combine the reference template data including, but not limited to, bioelectric impedance values, and retain these data in a form suitable for later retrieval. Portable local data storage may be used to manage reference templates of a small number of individuals, as might be with the use of storage located on a “smart” credit card. Stationary local data storage refers to a nonportable storage apparatus. This storage apparatus may be used to manage the reference template data for a larger number of individuals, as might be used either with bioelectric impedance data alone or in conjunction with additional identification means, e.g. a badge, key or numeric code, for a single door office access. Remote data storage refers to the storage of the reference template data at a location physically removed from an impedance measurement apparatus (reference and/or query). Remote data storage is anticipated to be used to manage reference data templates, e.g. a data management system, for identification systems utilized to identify a specific individual from a much larger group of individuals, or when the identification needs to be made at several different locations, as might be used for multiple location access, e.g., large building, ATM, gas station.

Many possible data storage methods exist depending on the specific requirement discussed above. Data storage methods (both portable and stationary) may include, but are not limited to, FLASH memory, Static RAM, Dynamic RAM, EEPROM, memory strips, magnetic tape, optical memory, e.g. CDROM, as well as more traditional means of data recording, e.g. hand written or typed notes recorded from displayed measurement data. For remote data storage, reference template data can be communicated to/from the remote location. There are numerous means to accomplish this communication including, but not limited to, the Internet, cellular phone, postal mail, transfer by computer disk, or by direct RF linkage. For those embodiments of the invention utilizing remote data storage, as well as in select variations of local or portable storage embodiments, the assessment may be accomplished either at the remote data storage location and the decision transmitted back to the location of local query measurement, or the reference data templates may be sent to the location of the query measurement for assessment. In yet another alternative embodiment of the invention, the remote reference template data and the query measurement data may be transmitted to a third location for the purpose of assessment, e.g. an access monitoring station.

As part of the transfer of query or reference template data from the measurement apparatus to other apparatus, e.g. the storage location, as well as in the storage of said data, one embodiment of the invention is to encrypt said data as well as other selected reference template data associated with the subject. Encryption diminishes the likelihood that unauthorized access or tampering with said data is achieved and thereby improves the overall integrity of the identification process. Methods for encryption are well known to those skilled in the art, and include but are not limited to, small encryption algorithms such as TEA (Wheeler and Needham, 1994) [TEA, a Tiny Encryption Algorithm, David Wheeler and Roger Needham, Computer Laboratory Cambridge University, November 1994] or more complex algorithms, e.g. SSL (Secure Sockets Layer) algorithms, such as those commonly employed to secure Internet communications. A logical choice for a key to such a encryption may be either a formula based upon the subject's name or other such readily obtained data, e.g. birth date.

### System Elements—Query

The identification of presumptively unknown subject as being included in the reference template database requires the obtaining of a bioelectric impedance values, or portion thereof, for comparison to the reference template records at times of identification need. Such obtaining of a subject's bioelectric impedance values is defined as the query (114).

In one embodiment of the invention, bioelectric impedance measurement apparatus for the purpose of query are located at or in close proximity to the identification need, e.g. a facility access need. Such apparatus locations include, but are not limited to, doorways of facilities, computer keyboards, “smart cards” (electronic cards such as proximity cards or contact cards having an embedded IC chip) used for the purpose of transactions or access, and on-body patches worn for the continual monitoring and wireless reporting of physiological status or access needs.

In alternate embodiments of the invention, bioelectric impedance measurements apparatus may be located remotely from the access need. Such embodiments may be useful to those subjects en route to an access location. The ability to remotely convey the query data (and/or assessment of query data with reference template records) may be desirable as well as time saving in certain circumstances, e.g. performing a query while driving up to a garage door and remotely performing the assessment process for accessing the garage while still approaching in a vehicle.

The query process is typically initiated by the activation of a bioelectric impedance measurement apparatus. In a preferred embodiment of the invention, activation of the measurement apparatus occurs when the subject comes into physical contact or is in close proximity to the measurement surface of the apparatus. Such activation of the system may be resultant from a variety of means, e.g. by a change in conductivity between measurement electrodes upon contact with the subject or an alteration of a light beam, acoustic signal, etc. which results in change in apparatus status from a resting state to one which is active. In alternate embodiments of the invention, the direct initiation of power to the circuitry of the apparatus, e.g. operation of an on/off switch, activates the apparatus for the purpose of query. In yet other embodiments of the invention, the query process occurs periodically or upon wireless command, such may be the case of an apparatus worn on-body or one that provides continual measurements.

Upon activation of a query bioelectric impedance measurement apparatus, the subject's bioelectric impedance values are obtained. The apparatus for obtaining a bioelectric impedance values for the purpose of query employs similar means of signal generation, electrical conduct elements, etc., as those described for the purpose of reference template measurement. In certain embodiments of the invention, the query apparatus may be the same apparatus used for obtaining reference template data. A key aspect of the invention is that, in general, the location on the body of the subject of the query bioelectric impedance measurement(s), e.g. the electrode contact locations, as well as the electrical signals, e.g. frequencies and currents, employed either duplicate those used in the creation of the reference template data or are correlate-able to the reference template data, e.g. by mathematical means, during the subsequent assessment process.

However, the electrical conductive elements, circuitry or overall apparatus employed for query bioelectric impedance measurements may be the same or differ in construction from those employed for collection of reference template data, e.g. the query apparatus may employ stainless steel electrodes whereas the reference apparatus employs gold coat material electrodes. The accuracy of the reference measurement may differ from that of the query measurement. In such circumstances, the assessment may compensate for this measurement difference.

In addition, the type or volume of bioelectric impedance data collected for the purpose of query may represent a subset of the data collected and stored in the reference template, e.g. only one signal frequency and current is utilized for the purpose of query whereas data from this frequency and current as well as data from several other frequencies and currents may be present within the reference template. Use of such subsets of electrical signals, e.g. frequencies, may represent a significant reduction in complexity in the overall design of query bioelectric impedance measurement apparatus while maintaining a high degree of security to the process, i.e. a subject intending to fool or trick the system may not know which frequencies may be employed and therefore will have to be prepared for all electrical signal possibilities.

Upon collection of the query bioelectric impedance data, the resultant data may either be assessed (compared to the reference template data) immediately without storage or stored for later assessment. Such data storage may also be for the purpose of recording and tracking identification queries, locations, etc.. If stored, the apparatus previously described for the transmittal and storage of the reference template data may be employed. It is understood that the transmission methods and storage utilized for reference template data may differ from that used for query, e.g. a reference template data may be stored on a CD ROM whereas the query data may be stored in flash memory.

### System Elements—Assessment

The third element of the bioelectric impedance identification system is that of assessment. Assessment (115) is defined as being the process either manual or electronic (plus associated software and hardware) whereby the concordance between one or more sets of query data and those present within the reference template data is determined.

The apparatus for electronic assessment, including hardware and software, is that of a comparator. Comparators may be constructed as stand-alone apparatus solely for the purpose of bioelectric impedance identification, having the requisite input and output features to communicate data and findings. In alternate embodiments, comparators may be comprise a portion of another apparatus, e.g. a personal computer workstation having the necessary communication methods and computational capabilities. Such apparatus for the storage and manipulation of databases and the operation of mathematical algorithms are well known to those skilled in the art of computer science. In use, the scope of this invention is not restricted any one embodiment or form of comparator.

Assessment may occur sequentially with the query activity or it may occur at a different time, utilizing stored reference template data and query data. In one embodiment of the invention, the assessment process occurs effectively instantaneously, e.g. within a period of a few seconds or less, of the query measurements.

In a one embodiment of the assessment, a first frequency query bioelectric impedance measurement or computed impedance value is compared to reference template data at that frequency (or to computed reference impedance templates based upon stored resistance and/or capacitance data). The comparison is based upon the degree of correspondence or numerical matching of the query values to those of the reference data templates. Such comparisons may have some degree of tolerance, e.g. +/−5% of the query value, being included in the assessment process. This degree of tolerance may be fixed or adjustable, including the ability to adjust the tolerance to the applied electrical signal (e.g. different frequencies or currents having different percentage tolerances) and/or adjusting the tolerance to the site on the subject's body of the measurement, e.g. hand as compared to torso. The output of the comparison is typically scored as being either yes or no, based upon the assessment of the query data and reference template data, e.g. a match or no match within tolerance.

It is assumed that one or more matches may be found in between the reference template database records and the query values, especially with large data base encompassing hundreds or thousands of subjects. These matches would correspond to the template data from one or more subjects. (If no match is observed, then the query may be rejected immediately or a signal to retry the query measurement is communicated to the query apparatus.) For the purpose of efficiency, the assessment algorithm can then restricted to the reference templates of those subjects identified in the first assessment. The query data from the next electrical signal/bioimpedance measurement frequency is then compared to the corresponding data of the selected individuals (or to computed impedance values based upon stored resistance and/or capacitance data). Those subjects whose reference templates are identified as matching (+/− tolerance) are then scored as a matching and the assessment process further restricted to these individuals.

The assessment process is repeated until all electrical signals and measurements from the query apparatus have been reviewed and at least one match observed in the reference template database, or until the determination of no match is made. If no match is found, the query may either be rejected or a command may be sent back to the query apparatus for remeasure of the subject and starting the query process over from the beginning. For re-measure/re-start loops of the query process, a finite number of attempts, e.g. three, may be made before the query is finally rejected.

Alternate embodiments of the assessment process, designed to either accept or reject a subject, are conceivable and may vary both to the sophistication of the algorithm/matching routine employed or the tolerances used. This scope of this invention is not restricted to any one particular embodiment of assessment algorithm or routine based upon the use of measured bioelectric impedance data or impedance computed from measured resistance and/or capacitance values.

As those skilled in the art of data analysis will recognize, there are two potential sources of error or “miscall” involved in such matching comparisons. The first error is that of false positive acceptance i.e. to accept query matches with a record template data incorrectly. The second is that of false negative rejection, i.e. to indicate a lack of match between query data and reference data when, in fact, the query subject is present within the reference data record. In order to minimize one or the other of these errors, tolerances in the analysis process may either be adjustable or fixed, thereby allowing a skilled practitioner to set levels of discrimination based upon the needs of the application.

Such comparative analysis tools are not restricted to the bioelectric impedance identification process and are readily available in a variety of formats, e.g. database comparison or one-to-one data comparison. Therefore, while one embodiment of the invention envisages analysis tools developed exclusively for reference template comparison, or towards a specific application utilizing bioelectric impedance identification, alternate embodiments of the invention may employ off-the-shelf comparative software to perform assessment tasks, e.g. database programs such as Access® from Microsoft, Inc..

In those embodiments of the invention wherein the query measurement is periodically activated, a third outcome may be observed, that of no query bioelectric impedance data (no signal) being obtained. This lack of bioelectric impedance signal (no signal) is then communicated for assessment and response. Such a situation may be observed if a query system intend for continual measurement is removed from the body of a subject or if, a periodic bioelectric impedance identification system employed to verify the subject is still in contact with the query apparatus does not detect the subject. The primary function of such periodic query apparatus may not be for the sole purpose of identification. Such apparatus may include, but are not limited to, electronic game controllers, electronic keyboards, heavy equipment controls to ensure the identity of the user while the apparatus is in extended use, or with “dead man” switches located on the equipment or vehicles where a hand grip or other form of conscious contact needs to be maintained to ensure safety.

In one embodiment of the invention, the outcome of the assessment is transmitted, displayed, stored or otherwise conveyed to a predetermined location or apparatus, i.e. the response. Such a location or apparatus may be at the site of the query, e.g. initiating the opening of a lock upon a door, or the outcome may be transmitted or conveyed to another, possibly distant, location or apparatus e.g. the recording the of the access of a database at a central monitoring facility located in a remote location, e.g. a different town, city or country. Such conveyance improves the utility of the invention and may be combined with other identification methods or apparatus as part of this activity to improve the overall effectiveness of the invention. In particular, the assessment may be coupled to other information associated with that subject, e.g. financial status, security or rank, and applied in combination with the bioelectric impedance data in the reference template data to modify or enable subsequent activities, e.g. open a door automatically.

In contrast to other identification methodologies such as fingerprint analysis or DNA identification analysis, bioelectric impedance measurements reflect the conductivity of the tissue, which is resultant from the tissue composition, e.g. the different cellular and fluid constituents, the degree of cellular and fluid heterogeneity, as well as the amounts and spatial distribution of these within the subject. This contrasts with an invariant, genetically determined attribute or feature, e.g. fingerprints. Therefore, bioelectric impedance measurement may be subject to variation. These variations may include but are not limited to, positional variation, diurnal variation, exercise or temperature related variation, or long term shifts in body composition due to diet, growth or exercise.

Site selection on the body for the electrical signal is one preferred means of minimizing potential variation in bioelectric impedance reading. Variation in segmental bioelectric impedance values are well known, with greater effects observed in the legs and lower torso, which are either positional or diurnal in nature. However, Zhu and coworkers report that segmental bioelectric impedance in the arm is not significantly affected by positional change, as compared to the leg. [Fansan Zhu, Daniel Schneditz, Erjun Wang, and Nathan W. Levin. “Dynamics of segmental extracellular volumes during changes in body position by bioelectric impedance analysis.” J. Appl. Physiol. 85(2): 497-504, 1998.] In a one form of the invention, bioelectric impedance measurements are made through segments or portions of segments of a body less affected by positional variation, e.g. hand to hand.

To provide additional data as a basis for compensation for possible variation in bioelectric impedance measurements due to exercise or other blood flow related changes in conductivity, the use of additional sensors, e.g. skin temperature or motion detectors, may be incorporated in some embodiments of the invention. In yet other embodiments of the invention, pattern or trend analysis to gauge daily or periodic variations in bioelectric impedance measurements may be incorporated into the assessment process. In still other embodiments of the invention, additional bioparameters, e.g. weight, may be added to provide additional mathematical means to improve the accuracy of the assessment process.

To extend the overall useful lifetime, e.g. the time between re-measuring subject's data for inclusion or updating of the reference template data, one embodiment of the invention is to provide trend analysis of a subject's bioelectric impedance template over an extended time, e.g. days or weeks. That is, in one embodiment of the invention, successive measurements are periodically obtained, e.g. a first set plus at least one additional set of reference measurements, providing the basis for subsequent trend analysis on the measured values of the bioelectric impedance from one or more subjects. It is anticipated that these variations or trends may be detected without accepting or rejecting a subject. If such a trend is detected, the reference biometric template may have additional factors applied, e.g. modified, such that acceptance of the input query value is maintained for a period greater than that achieved in the absence of such added factors.

In a related embodiment of the invention, such repeated measurements are obtained as repeated reference bioelectric impedance measurement sessions taken over time, e.g. days, weeks or months. Such multiple reference data sets, e.g. a first set plus at least one additional second set, plus the possible addition of other factors, e.g. weight, time of day, month, eating patterns, exercise patterns, etc., provide the basis for subsequent algorithm development and/or allow artificial intelligence pattern definition techniques to be employed. Such multivariable trend analysis methods are well known to those skilled in the art of mathematics. For example, if diurnal patterning of a subject indicated a predicted, e.g. >90% likelihood, of a decrease, e.g. 3% decrease in a bioelectric impedance value in the late afternoon, e.g. 4 pm, as compared to early morning, e.g. 8 am, then reference template data values may be adjusted or modified by interpolation through an assessment algorithm to the time of day to reflect a predicted change. That is, at noon the reference template data value would be modified to a value 1.5% higher than the reference template data value employed in late afternoon, and 1.5% lower than the morning value, assuming a linear interpolation of the data. Such adjustments, if required, have utility for activities with predictable or habitual use, e.g. daily access at a factory or to a computer workstation.

In yet another embodiment of the invention, query data may be included in reference template data sets and utilized for subsequent adjustment algorithms. One example of this process is, if during the query/assessment process, such query data is accepted by other means, e.g. by command given to the system by an individual in authority, and therefore may be included into the reference bioimpedance data record. Such data may permit adjustable modification of the system such that a subject may be provide the basis, e.g. on a weekly or monthly basis, of updating and adjusting the reference template data to accommodate changes in lifestyle, e.g. dieting, and/or occupation.

In an alternate embodiment of the invention, the date and time of the query is noted and compared to that of the reference template measurement. Such measurements may be associated with predictive changes based upon formula derived from a larger number of subjects. Such changes may include changes in body hydration or dietary profiles, e.g. patterned diurnal or menstrual variation, and the assessment algorithm adjusted according to either elapsed time or time of day to minimize the risk of false rejection or acceptance.

In yet another embodiment of the invention, adjustment for differences in electrical path lengths between one or more reference and/or query measurements may be made. That is, in measurement, slight variations in contact points with electrically conductive elements caused by flexing of the hand, fingers, etc., may alter the electrical path length or route. Such positional variations may be recognized by either manual or automatic sensing or detection means, and the results of these positional variations may be incorporated as part the query and assessment processes. In such embodiments of the invention, the query bioelectric impedance values may be adjusted prior to or as a part of the assessment with the reference template data records. For example, consider positional variations of intra-hand measurements. With the assumption that bioelectric impedance measurement are linear (or otherwise mathematically describable) between the electrically conductive measurement elements, then the measured bioelectric impedance may be adjusted to accommodate for differences in the query contact lengths or differences in distance between contacts. It is understood that such adjustments may introduce an inaccuracy in the modified query value but, in certain applications, such an inaccuracy may afford a greater degree of utility to the invention than an unadjusted measurement.

The completion of the assessment process includes the determination of match (or non-match) between a query and the reference template data The determination may or may not include additional information, such as, but not limited to, the degree of confidence or uncertainty of the strength of the match and the number and types of possible matches within the reference template data. The completion of an assessment may be coupled to a presentation of a determination, e.g. a response. Such response may include, but is not limited to: no response; providing or enabling access to a facility, equipment, records or systems; activation of a light (e.g. LED or organic light emitting diode) or display (e.g. liquid crystal diode display or cathode ray tube display) indicative of assessment outcome, (e.g. green equating to identification confirmed, red equating to identification not confirmed); transmittal of the query/assessment process to a third party involved in monitoring identification activities; initiating a pause or stoppage of ongoing electronic or mechanical activities (automatic interrupt), etc. The latter response is favored for those periodic query systems wherein the query apparatus is also a computer keyboard, equipment control, etc..

In one embodiment of the invention, the assessment response initiates a series of activities, including but not limited to, either permitting or denial of access to the requested system, facility, equipment or apparatus, e.g. to financial records, computer systems, equipment or facilities. The subsequent action that results from this assessment may be controlled by the identification system or be interconnected with an access control device by various fashion, e.g. wired or wireless.

### Use in Combination With Other Identification Means

In certain applications, bioelectric impedance identification may be employed in combination with other identification methods, e.g. proximity identification cards, fingerprint scanning, hand morphology scanning, iris scanning, voice recognition, code words/numbers, etc.. Combination or layering of bioelectric impedance identification systems with one or more other additional methods of identification may improve the overall identification process. In use, such combinations may be within apparatus constructed to enable bioelectric impedance query measurements with one or more methods of identification, e.g. a fingerprint scanner, or the bioelectric impedance data may be subsequently combined with data obtained from one or more separate and distinct apparatus for these other forms of identification to form a more robust reference template for the purpose of assessment.

The use of a combination of methods for identification may afford improved identification stringency (reduced likelihood of false acceptance miscall) over a single means. For instance, a fingerprint identification method may provide 95% accuracy (a “miscall” rate of 1 out of 20). Likewise, a bioelectric impedance identification may provide 95% accuracy with similar miscall rate. A predicted improvement of identification stringency by utilizing the two methods together may be considered being the product of error rate of the two processes, which results in a combined accuracy of 99.75% (a “miscall” rate of 1 out of 400), assuming the independence of the two methods.

One skilled in the art will readily appreciate that a variety of weighting or factoring techniques may be applied to improve the identification process utilizing two or more different methodologies. Therefore, one preferred embodiment of the invention is the combination of bioelectric impedance identification with one or more additional means of identification to thereby improve the overall identification process. Such additional means of identification include, but are not limited to, fingerprint analysis, iris pattern recognition, facial morphology, alphanumeric code entry, RF identification cards or tags, weight, height, DNA identification analysis and voice recognition.

Dependent upon the identification need or application, the degree of acceptable miscall may vary. For instances, when multiple identification means are employed in addition to the bioelectric impedance means described herein, the accuracy of any one means, such as the bioelectric impedance identification, may be as low as 1 out of 2, i.e. identification of a subject as being as member of half of the reference data base templates (not rejected). Such a low rate when multiplied by other, preferably independent, identification means may provide sufficient improvement of the overall identification process to provide utility in the application. An example of such a useful combination would be the bioelectric impedance identification plus RF identification card (a non-biometric based form of identification and readily transferable) increasing the likelihood that the holder of the card is the subject issued said card.

An additional embodiment of the invention is the use of bioelectric impedance identification with the use of an apparatus, one which may be worn or affixed to the body containing the means to respond to an inquiry signal. This inquiry signal may be a part of the bioelectric impedance measurement signal or an additional signal, including but not limited to, electrical, RF, acoustic, or optical signals. The response of this apparatus to the inquiry signal may include the release of stored information. Stored information may include, but is not limited to, serial number of the apparatus, biometric data (weight, height, facial features, etc.), or stored bio-impedance values. The means of receiving and transmitting signals to and from the apparatus may include, but is not limited to, electrical, radio, acoustic, infrared or mechanical means.

In one example, an electrical inquiry signal is sent from the bioelectric impedance electrodes in contact with a surface of the body and is received by an apparatus, e.g. a patch containing the necessary memory and control circuitry, power and electrical connections (electrodes) located on the body using electrodes on the patch to pick up the signal. The signal frequency(s) chosen may be the same or a different frequency than those of the bioelectric impedance signal(s) sent through the subject to generate the bioelectric impedance data. Transmitted responses from the patch may include, but are not limited to, the ability to transmit a response signal, e.g. a signal based upon the timing of the received bioelectric impedance signal, or an identifying code, or the sending additional data useful for identifying the individual, e.g. facial image, height, weight. Such a transmission means may include but is not limited to, acoustic, infrared, RF or visible light means.

Sending a response to the introduced inquiry signal may be useful in assuring that the apparatus has remained with the original subject and has not been given to or placed on another subject. That is, any alteration of the introduced signal characteristics, e.g. response to signal by degree of phase shift, resultant from removing the apparatus and either placing it on a different individual or in a different location may be detectable, as compared to initial set-up data values. In addition, the apparatus may send an identification code, encrypted or in plain text, in response to the inquiry signal. This feature may include encryption key, randomization codes or other features to aid in providing a secure identification metric to the query apparatus.

### Supporting Bioelectric Impedance Data

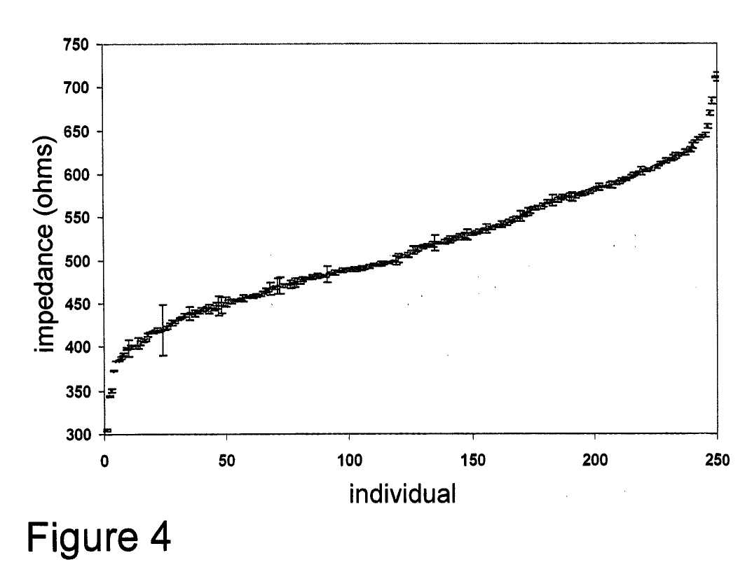

FIG. 4 shows impedance (resistance) in ohms of 250 subjects measured from foot to foot at 20 KHz, ranked from lowest to highest. Each individual was independently measured in triplicate (+/− standard deviation). This data shows that impedance measurements at a single frequency measurement by itself offers a level of discrimination or identification among individuals. That is, for any one subject, there may be 40 other subjects with similar impedance values. This provides a limited degree of biometric identification capability, e.g. (250-40)/250=0.84 or 84% of the population would be ruled out by this simple test.

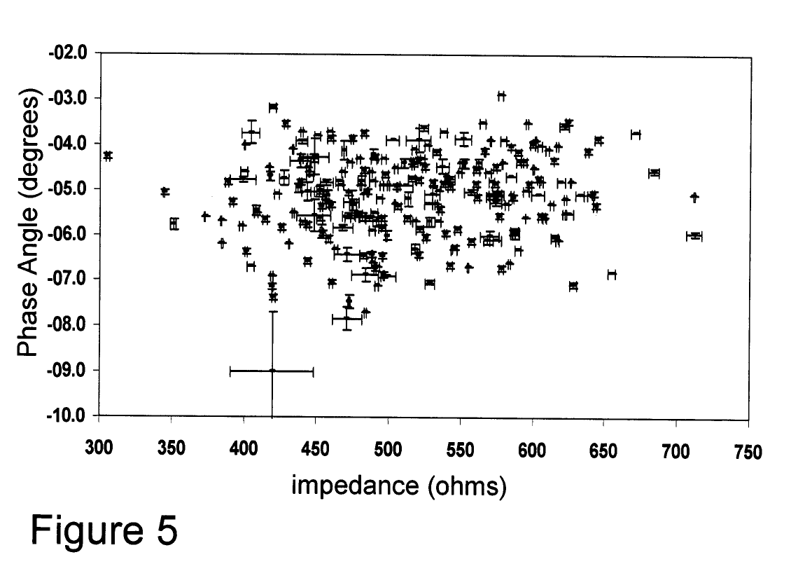

Discrimination between subjects is improved by including additional bioelectric impedance data parameter, i.e. phase angle also at 20 KHz, (FIG. 5). In this figure, each subject's phase angle (+/− standard deviation) is plotted against their impedance (+/− standard deviation). In this example, impedance is an independent parameter from phase angle (r² =0.016) and therefore measurement of both offers improved identification to as compared to impedance values alone. For example, if impedance alone passes or accepts 16% of the population and similar percentages are observed with phase angle, then a combination of impedance and phase angle would reject 97.5% (accept 2.5%) of the tested population.

### EXAMPLES

Uses and applications of bioelectric impedance identification system include, but are not limited to, uses involving providing identification for the purpose of local or remote access, e.g. to facilities, garage doors, electronic systems, such as computers, databases or games, or for the purpose of identification of individuals for the purpose of recognition, e.g. use with “smart” credit cards, as players of electronic games, or as part of remote body health monitoring systems. Although the examples below are indicative of the type of uses the bioelectric impedance identification system can be applied to, they are not meant to limit the scope of the invention. Those of ordinary skill in the art can appreciate the many applications that the bioelectric impedance identification system may be used in, and with no undue modification of the system, may be adapted to.

### Example 1

Use of Bioelectric Impedance Identification to Permit Access to a Facility

One application of bioelectric impedance identification may be the routine use to provide employees access to company facilities, such as allowing access through doorways from the outside to inside of the building or within the building itself. One possible method by which this may be accomplished with this invention would be that reference bioelectric impedance template data would be obtained from an employee upon their enrollment with the company and stored in the reference template database. Said reference bioelectrical impedance data may be obtained using a table top bioelectric impedance apparatus, comprised of electrodes and circuitry designed to obtain bioelectric impedance data from several points on one hand. The reference bioelectric impedance data would then be communicated through a wired link to the reference template data management system within the company's data system for storage and retrieval upon command.

Hand query bioelectric impedance measurement apparatus maybe located adjacent to doorways where secure access or control accessed is desired. The employee desiring to pass through the doorway would walk up and, using same hand as used for reference template creation, have their bioelectric impedance query measurement taken. An example of such a interface to obtain query bioelectric impedance template measurements is illustrated in FIG. 3 which indicates the position the user should place their hand (205), guide posts for positioning their hand repeat ably (208,209) and the signal source (201,203) and measurement (202, 204) electrodes. This query measurement would then be transmitted for assessment back to the company's data system. If a match is found and permitted, i.e. that employee is authorized to access that particular area, a command will be transmitted to the latch system of the door, thereby opening the door. In addition, a light may be turned on, e.g. green light, indicating to the employee the assessment process identified them and permits access.

As one of ordinary skill can readily appreciate, the above use of the identification process can be expanded to include tracking of the employee's activities throughout the building, automatic change of access privileges, etc., and also that this system may be incorporated with other identification methodologies commonly employed in industry, e.g. RF tagged identification badges or keypad access. The advantage bioelectric impedance systems offer over such systems is that the identification is non-transferable, that is, an employee cannot pass their bioelectric impedance values to another individual whereas they can do that with a RF identification badge. In addition, one can readily appreciate that such bioelectric impedance technology may be incorporated into other biological parameter based identification technology, such as hand geometry scanners, to improve the robustness and accuracy of the identification process. Finally, one can readily appreciate that such a system may be adapted to incorporate repeated reference data repeatedly updated by additional reference data sessions, e.g. daily or weekly, to track trends in parameters affecting bioelectric impedance values, e.g. body composition changes, dietary changes, etc..

### Example 2

Use of Bioelectric Impedance Identification to Permit Access to a Computer Workstation

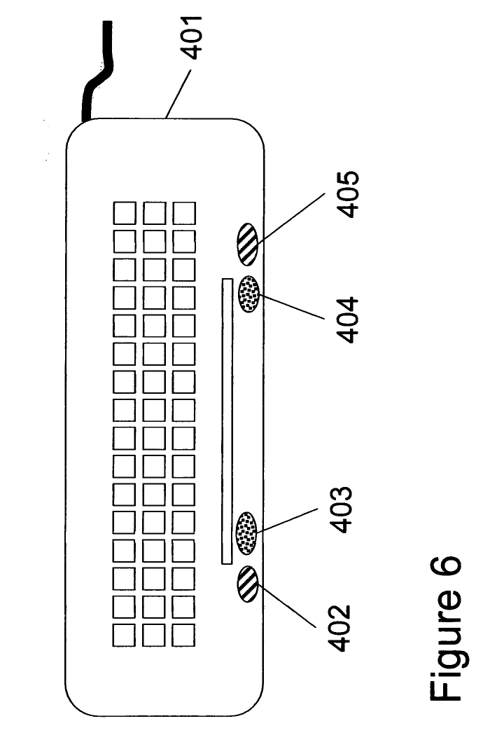

In this example the bioelectric impedance reference template and query apparatus are located within a personal computer (PC) workstation. The bioelectric impedance system also includes necessary software and instructions to enable the set up of the system within the PC workstation and to allow subsequent assessment of local queries. In use, a computer user first sets up the system by recording their reference bioelectric impedance template by following a series of instructions provided as part of the system. Electrodes for bioelectric impedance use may be incorporated as part of the keyboard, e.g. with signal introduction electrodes designed for contacting the index finger of each hand when at standard rest on the keyboard and the measurement electrodes contacting the middle finger of each hand. In this example, the standard QWERTY keyboard would have these fingertips normally resting on the F, J and D, K keys respectively and therefore may be considered logical electrode locations. Other variations, e.g. contacts on the edge of the keyboard, etc., are readily conceivable. For example, FIG. 6 wherein the keyboard (401) has both signal introduction electrodes (403,404) and measurement electrodes (402, 405) mounted on the frame beneath the touch key pads. Upon fingertip contact with the electrodes, the bioelectric impedance measurement system would automatically be activated and reference bioelectric impedance measurements obtained.

Subsequently, the computer user may simply turn the computer on and place their fingertips on the electrodes. The query bioelectric impedance measurement would be obtained and compared to reference template data. If the user is recognized i.e. a match is obtained between reference template data and the query data, access to the computer system, configuration files and Internet connections may then be enabled automatically, as specified by the initial set up of the system for that user. Such a system will permit easier use of computer systems, avoiding the need to repetitively type in passwords throughout the day. As can be readily appreciated by one of ordinary skill, such a system may be readily adapted to a number of related applications. Such applications include the permitting of multiple users of a single PC workstation, the periodic validation that the user of the workstation is authorized by the use of a periodic query measurement, i.e. that a non-authorized individual is not operating an already functioning PC workstation or the recording and relaying of the workstation use and user identity for system maintenance and administration, etc..

### Example 3

Use of Bioelectric Impedance Identification to Permit Access to a Computer Gaming System

Related to the previous example where a bioelectric impedance identification system provides access to a computer workstation, bioelectric impedance identification may be applied to electronic apparatus such as gaming stations, such as the Sony Playstation®, Nintendo GameBoy®, Microsoft Xbox®, etc.. The set up software may be installed in the factory or subsequently installed into the gaming station by the user. The necessary hardware is incorporated in to the main gaming box or into associated input/output (I/O) control units. In particular, the electrodes to provide user identification may be incorporated into I/O control unit, e.g. as part of a joystick (one hand reading) or control pad (between two hand readings). To set up the system, the user would follow instructions displayed on the game screen, recording both their bioelectric impedance template as well as an identifying name. In use, the user would turn on the gaming system, hold the control unit and the system automatically identifies the user. Such information as the activation of the game previously played, return to last move made in the previous game, overall gaming statistics and scores, etc., might then be automatically be presented to the user, dependent on the set up and instructions. If the user is not identified, the system may return to default (standard) interfaces and controls.

Such an automatic bioelectric impedance identification system would facilitate scoring and record keeping for users, and provide simpler, easier access for users, especially a multiple locations, e.g. internet based gaming. As can readily be appreciated, such a bioelectric impedance system may be readily adapted to include other capabilities, e.g. including the use of feedback lights indicative of electrode contact quality as well as incorporating automatic pause/off commands if the controller is set down. Such a means would be enabled if a periodic bioelectric impedance measurement signal were sent through the electrodes to obtain bioelectric impedance measurements. If no signal was observed, an outcome of the assessment process may be to pause or terminate the game. Such a feature may also have a related utility in the previous example, Example 2—Access to Computer Workstation, whereby the computer may enter a power savings mode if not in use for a period of time.

### Example 4

Use of Bioelectric Impedance Identification With a “Smart” Credit Card

One portable use of bioelectric impedance template is the incorporation of a user's bioelectric impedance reference template as part of the information stored on a “smart” credit card, i.e. within the electronic chip contained within the card. In this case the signal introduction and measurement electrodes may be located on opposing surfaces of the card or they may be located on the same surface. The user contacts the electrodes with both hands, e.g. holding the card between the thumb and forefinger of both hands. and the circuit formed passes through both hands and the upper torso of the user. The bioelectric impedance reference template data stored in the cared may either be incorporated prior to the card being sent to the user, i.e. the user's data is entered via a reference bioelectric impedance apparatus located at a bank or other financial institution, or the reference data is recorded as part of the card activation process by the user, i.e. upon receiving the card, the user following a series of steps causes a card to become active for recording. Such steps may include activating a photo switch then grasping the card for a predetermined period indicated by visual signal (e.g. organic light emitting diode display), which indicate the quality of contact and the bioelectric impedance measurement status. Once a reference template has been stored on the card, the user may present the card at a place of commerce, and by grasping the card, the validation of that the user holding the card is the same as the one who provided the reference data is indicated. Such indications may be by a visual display, using the same display technology used to validate entry of reference bioelectric impedance template data.

Such identification technology incorporated into a “smart” card format may be readily expanded to include the use of other technologies, e.g. RF links, transmittal of additional information upon card reading including financial data or additional identification information, such as fingerprint data, for use with the card. Additional applications also may include the use of reference template data stored remotely (and not on the card). Upon use, e.g. during a first financial transaction, the query data from the card would then be transmitted to the location of the reference template database for subsequent assessment.

We claim:

1) The method of bioelectric impedance identification comprised of the steps of:
a. introducing, for the purpose of creating a first reference template data set, one or more electrical signals into a subject's body via a plurality of electrically conductive structures at one or more locations on the body;
b. measuring by a plurality of electrically conductive structures the electrical impedance resultant from said first set of introduced electrical signals;
c. storing said first set of measured impedance resultant from said first set electrical signals introduced for the purpose of reference template creation;
d. introducing, measuring and storing at least one subsequent additional set of electrical signals for the purpose of modification of the reference template;
e. adjusting said first reference template bioelectric impedance values by impedance values forthcoming from one or more said additional sets of measurements;
f. introducing for the purpose of query one or more electrical signals via a plurality of electrodes into a subject's body at one or more locations;
g. measuring by a plurality of electrically conductive elements the electrical signals introduced for the purpose of query;
h. comparing for the purpose of assessment one or more measured bioelectric impedance values resultant from one or more electrical signals introduced for the purpose of query to one or more adjusted reference template bioelectric impedance values.
i. presenting the outcome of the assessment.

2) The method of claim 1 where said additional reference measurements are taken on a periodic basis.

3) The method of claim 2 where said periodic basis is more than once a day, daily, weekly or monthly.

4) The method of claim 1 where one or more of said additional reference measurements are comprised of bioelectric impedance data sets resultant from query measurements.

5) The method of claim 1 wherein the electrical signal introduced is from between the frequency of 100 Hz to 1 MHz.

6) The method of claim 5 wherein the electrical signal introduced is preferably between the frequency of substantially 5 KHz and substantially 250 KHz.

7) The method of claim 1 further including the step of measuring one or more additional means of identification and utilizing that identification in assessment.

8) The method of bioelectric impedance identification comprised of the steps of:
a. Introducing, for the purpose of creating a reference template data set, one or more electrical signals into a subject's body via a plurality of electrically conductive structures at one or more locations on the body;
b. measuring by a plurality of electrically conductive structures the electrical impedance resultant from said introduced electrical signals;
c. storing said measured impedance resultant from electrical signals introduced for the purpose of reference template creation;
d. introducing for the purpose of query one or more electrical signals via a plurality of electrodes into a subject's body at one or more locations;
e. measuring by a plurality of electrically conductive elements the electrical signals introduced for the purpose of query;
f. comparing for the purpose of assessment one or more measured bioelectric impedance values resultant from one or more electrical signals introduced for the purpose of query to one or more recorded reference template bioelectric impedance values;
g. and presenting said assessment.

9) The method of claim 8 further including the step of measuring one or more additional means of identification and utilizing that identification in assessment.

10) The method of claim 9 wherein such additional means of identification comprise fingerprint analysis, iris pattern analysis, facial geometry analysis, keypad code entry and identification badges.

11) The method of claim 8 wherein the electrical signal introduced is from between the frequency of 100 Hz to 1 MHz.

12) The method of claim 11 wherein the electrical signal introduced is preferably between the frequency of substantially 5 KHz and substantially 250 KHz.

13) A system of bioelectric impedance identification including:
a. an apparatus for the generation and measure of a first set of reference bioelectric impedance arising from the introduction of one or more electrical signals into a body;
b. a storage of said first set of measured impedance resultant from said first set electrical signals introduced for the purpose of reference template creation;
c. an apparatus for the generation and measure of at least one additional second set of reference bioelectric impedance arising from the introduction of one or more electrical signals into a body;
d. a storage of said second additional set of measured impedance resultant from said second set of electrical signals introduced for the purpose of reference template creation;
e. an adjustor of reference bioelectric impedance values generated by a first set reference measurements by subsequent values from said additional set of reference measurements;
f. an apparatus for the generation and measure for the purpose of query of bioelectric impedance arising from the introduction of one or more electrical signals into a body;
g. a comparator comparing for the purpose of assessment one or more measured bioelectric impedance values resultant from one or more electrical signals introduced for the purpose of query to one or more modified reference bioelectric impedance values.

14) The system of claim 13 wherein said additional measurements are taken on a periodic basis.

15) The system of claim 14 wherein said periodic basis is more than once a day, daily, weekly or monthly.

16) A system of bioelectric impedance identification comprising:
a. an apparatus for the generation and measure, for the purpose of reference template creation, of one or more electrical signals into a subject's body via a plurality of electrically conductive elements at one or more locations on the body;
b. a storage of said measured impedance resultant from electrical signals introduced for the purpose of reference template creation;
c. an apparatus for the generation and measure, for the purpose of query, of one or more electrical signals via a plurality of electrodes into a subject's body at one or more locations;
d. a comparator to compare for the purpose of assessment one or more measured bioelectric impedance values resultant from one or more electrical signals introduced for the purpose of query to one or more recorded reference template bioelectric impedance values.

17) The system of claim 16 further including the apparatus and systems for measuring one or more additional means of identification and utilizing that identification in assessment.

18) The system of claim 17 wherein such additional means of identification comprise fingerprint analysis, iris pattern analysis, facial geometry analysis, keypad code entry and identification badges.

19) The system of claim 16 wherein the electrical signal introduced is from between the frequency of 100 Hz to 1 MHz.

20) The system of claim 19 wherein the electrical signal introduced is preferably between the frequency of substantially 5 KHz and substantially 250 KHz.
